(19) United States
(12) Patent Application Publication
(10) Pub. No.: US 2005/0216048 A1
(43) Pub. Date: Sep. 29, 2005

(54) Surgical tool for treating varicose veins

(76) Inventors: William D. Suval, Apple Valley, CA (US); Lev Melinyshyn, Buffalo Grove, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/806,641

(22) Filed: Mar. 23, 2004

(51) Int. Cl. A61B 18/18; A61M 29/00

(52) U.S. Cl. 606/194

(57) ABSTRACT

A surgical tool for extracting varicose veins includes an elongate handle with a hook at one end and a piercing/dilating head at the other end. The piercing/dilating head is bulbous in shape and has both a sharp tip and a dilating surface that can be used to dilate an opening without making a longitudinal incision. The hook can be used to extract the vein through the dilated opening. Sculpted sections on the piercing/dilating head help to prevent the tool from rolling on flat surfaces, and alternating sections of relieved surfaces and surfaces with polygonal cross-sections make it easier to grip.

### BACKGROUND OF THE INVENTION

1. Field of the Invention

The present invention relates generally to medical treatment, and more particularly to a surgical tool adapted for use in puncturing tissue and treating varicose veins.

2. Description of Related Art

Varicose veins are a medical condition present in up to twenty-five percent of the adult population, and are especially prevalent among middle-aged women. The term “varicose” is derived from the Greek word for “grape-like” and refers to the tortuous appearance of the afflicted veins. Patients suffering from varicose veins often experience various symptoms including aching, itching, heaviness, swelling or cramping of the legs, while more serious complications can include thrombophlebitis, dermatitis, hemorrhage and ulcers. Even absent such complications, many patients seek medical treatment of varicose veins for primarily cosmetic reasons due to the generally unsightly appearance that characterizes the condition.

Specifically, varicose veins are a condition of the superficial veins of the legs in which the veins have become abnormally twisted, lengthened or dilated. Inefficient or defective one-way valves within the veins usually cause the condition. These one-way valves provide an important function in controlling blood pressure within the venous system of the legs. During walking, the leg muscles provide a musculovenous pump that compresses the veins and propels blood to the heart. Efficiency of the musculovenous pump is enhanced by the one-way valves within the veins that protect the venous system at the lower extremities from excess pressure generated by coughing, straining, lifting, standing or other such exertion. The superficial veins normally carry only ten to fifteen percent of the blood, with the remainder being carried by the deep veins; however, the percentage of blood carried by the superficial veins can exceed these normal levels due to dilation of the superficial veins or thrombosis of the deep veins. As a result, the one-way valves can become incompetent, which further increases retrograde pressure within the superficial veins. Since the superficial veins lie close to the skin layer and are poorly supported by the subcutaneous tissue, the increased retrograde pressure causes the varicose veins to be formed.

There are many methods used for treating varicose veins. Selected methods vary depending upon the characteristics of the vessel to be treated and the preferences of the physician performing the procedure. These methods include surgical ligation and stripping, ambulatory phlebectomy, sclerotherapy, radiofrequency (RF) occlusion, laser occlusion, transilluminated powered phlebectomy and electrodessication.

With the ligation and stripping technique, a surgeon first makes an incision at the groin area through which the saphenous vein is separated from the femoral vein. The saphenous vein is also dissected at the foot end of the saphenous vein. The surgeon then uses a stripping device to extract the vein. Once the saphenous vein is completely removed from the leg, the various incision wounds can be sutured closed.

The stripping technique represents a permanent solution in that the varicose vein condition cannot recur once the vein has been removed. Nevertheless, the technique has numerous significant drawbacks. The numerous incisions often leave substantial unsightly scars along the legs that can be as unpleasant in appearance as the original varicose vein condition. Moreover, the procedure is generally performed under general anesthesia and often requires an overnight hospital stay. There are also associated complications of the technique, such as blood loss, pain, infection, hematoma, nerve injury and swelling. After undergoing the stripping technique, a patient generally requires several weeks to recover. In view of these significant drawbacks, the stripping technique is recommended only for extreme cases of varicose veins, and for patients that are in sufficiently good health to handle this type of surgery.

Sclerotherapy involves injection of a toxic fluid, such as sodium tetradecyl sulfate, into the veins to cause subsequent inflammation and sclerosis of the veins. The sclerosis results in localized scarring or closure of the veins, which forces rerouting of the blood away from the affected veins. The sclerotherapy technique is often combined with an operative procedure, such as ligation of a portion of the saphenous vein.

While the sclerotherapy technique is less surgically intensive than the stripping technique, it often does not represent a permanent or complete solution since it has a high rate of recurrence and cannot be applied to the saphenous vein in the upper thigh region due to the risk of sclerosis of the deep veins. Sclerotherapy has other potentially serious complications including skin staining, ulceration, phlebitis, allergic or anaphylactic overdose, ischemia, skin or fat necrosis, and peripheral neuropathy. Notwithstanding these complications, patients must often undergo multiple courses of sclerotherapy treatment in order to completely alleviate the varicose veins to a satisfactory degree.

RF occlusion, laser occlusion, and transilluminated powered phlebectomy are not well suited for smaller vessels and require substantial investments in capital equipment.

In view of these significant drawbacks, a critical need exists for a surgical apparatus that would reduce the invasiveness and associated recovery time of the conventional treatment for varicose veins.

Ambulatory phlebectomy (AP) is a particularly attractive procedure of choice because, in contrast to other options, it can be performed under local anesthesia on an ambulatory basis. Furthermore, it requires only very small micro-incisions which are cosmetically pleasing to the patient and the surgeon.

A Swiss dermatologist, Robert Mueller, M.D., developed AP in 1957. As with many new procedures, his colleagues criticized Dr. Mueller because he did not use large incisions to find veins.

With this surgery, tiny incisions are made in the vein, and then with surgical hooks, the vein is pulled out of the leg. The vein usually is removed in one treatment. Compared to traditional surgery, ambulatory phlebectomy allows the removal of very large varicose veins while leaving only very small scars. Patients can return to normal activity the day after treatment.

AP is presently performed by making a small skin incision near the vein to be removed and by then using a phlebectomy hook to engage and remove the vein. Incisions are made using conventional surgical scalpels. The hooks that are presently used come in various sizes and configurations and are designed to be reusable. They are manufactured from stainless steel and are cleaned and sterilized after each use. Commonly used reusable hooks include the Mueller, Tretbar, Oesch, Ramelet and Varady hooks. The currently available hooks do not have the ability to make the skin opening.

In U.S. Pat. No. 5,758,665 to Suval, an apparatus is described which forms an incision and extracts the vein. This invention uses a scalpel edge to make a longitudinal incision. Similarly, in U.S. Pat. No. 5,792,168 to Suval, an apparatus is described which forms an incision and extracts the vein. The apparatus also uses a scalpel edge to make a longitudinal incision. In an alternative embodiment, a gun-shaped mechanism is used to extract the vein.

### SUMMARY OF THE INVENTION

Advantages of the present invention can be achieved using an improved surgical tool for AP.

The tool disclosed herein includes a hook that can be used to engage and remove the unwanted varicose vein. Unlike previously-known devices, the tool includes a unique means for making micro-punctures for hook passage and vein extraction. Unlike the invention disclosed in the Suval patents identified above, for example, the tool disclosed in this application makes no incision. Rather, it makes a small hole and then dilates it to a larger diameter.

In one embodiment of the invention, the tool has a handle with a hexagonal cross-section. The handle has a number of relieved sections along its length. The hexagonal shape together with the relieved surfaces gives the handle excellent gripping characteristics.

A bulbous piercing/dilating head is mounted on one end of the handle. The head is used to first pierce the skin. The unique shape then dilates the opening as the head is advanced to a size that will allow subsequent passage of the hook. The shape of the head prevents the sharp tip from penetrating too deeply into the tissue.

A “J” shaped hook can be mounted on the other end of the handle. Properly sized, the hook can be passed through the hole made by the piercing/dilating head and used to engage and remove the varicose vein.

A more complete understanding of the tool for treating varicose veins will be afforded to those skilled in the art, as well as a realization of additional advantages and objects thereof, by a consideration of the following detailed description of the preferred embodiment. Reference will be made to the appended sheets of drawings which will first be described briefly.

### BRIEF DESCRIPTION OF THE DRAWINGS

FIG. 1 is a front view of a surgeon using the piercing/dilating head of the tool to make a skin opening;

FIG. 2 is a side view of a surgeon using the hook of the tool to engage the varicose vein;

FIG. 3 is a side view of a surgeon using the hook of the tool to remove the varicose vein;

FIG. 4 is a side view of the surgical tool;

FIG. 5 is an enlarged side view of the piercing/dilating head;

FIG. 6 is an end view of the piercing/dilating head seen in FIG. 5;

FIG. 7 is cross-sectional view taken through lines 7-7 in FIG. 5;

FIG. 8 is an enlarged side view of the hook; and

FIG. 9 is an enlarged partially-rotated side view of the piercing/dilating head seen in FIG. 5.

### DETAILED DESCRIPTION OF A PREFERRED EMBODIMENT

The present invention may satisfy the need for a surgical tool that can reduce the invasiveness and associated recovery time of the conventional treatment for varicose veins.

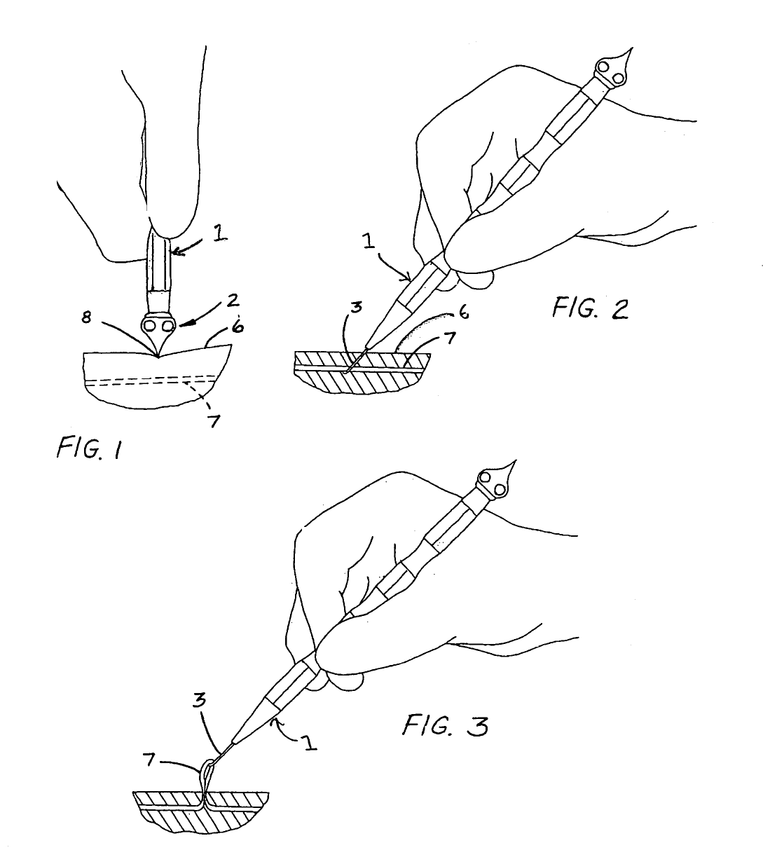

FIG. 1 is a view of a surgeon using a tool 1 that forms one embodiment of the present invention. The surgeon is using the piercing/dilating head 2 of the tool to make a skin opening 8 near the superficial varicose vein 7 to be extracted. The superficial vein 7 lies between layers of subcutaneous tissue and sartorius muscle. The specific segment of vein 7 requiring treatment may comprise the saphenous vein or one of its tributary veins, depending upon the specific condition of the patient. Prior to treating the specific vein segment, the saphenous vein may be disconnected from the femoral vein, as known in the art.

FIG. 2 shows the surgeon using the hook 3 of the tool to engage the varicose vein 7, and FIG. 3 shows the surgeon using the hook 3 of the tool to remove the varicose vein 7.

In accordance with the present invention, a single tool can be used to create a small opening through the skin layer 6 and to engage the vein 7 for extraction through the opening. Once extracted, the superficial vein may be ligated using known surgical techniques. Subsequently, the hole through the skin layer 6 can heal normally and, by keeping the hole small, scarring of the skin can be minimized. Moreover, it may be possible to perform the procedure on an out-patient basis without any of the usual complications of conventional surgical procedures.

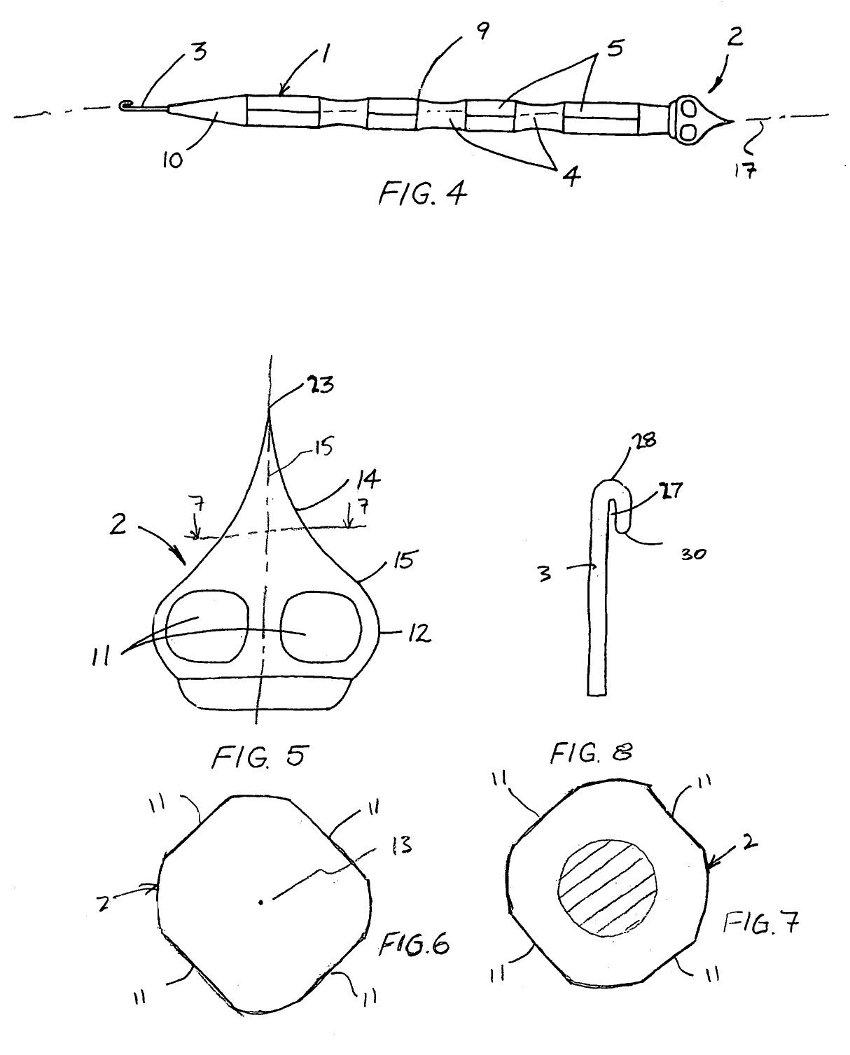

As shown in FIG. 4, the tool 1 that has been illustrated here comprises an elongate handle 9 that has relieved surfaces 4 that alternate along the length of the handle with surfaces 5 that have polygonal (in this case hexagonal) cross-sections. These surfaces provide excellent gripping characteristics even when the tool is wet and held in a surgically-gloved hand. The illustrated handle 9 is substantially cylindrical and can be made from any commonly-used medical grade material. These materials would include plastics like nylon, ABS, or PVC; or a metal like stainless steel.

In the illustrated embodiment of the invention, the piercing/dilating head 2 has a bulbous shape and is mounted on one end of the handle 9. The hook 3 is mounted on the other end of the handle. The portion of the handle that holds the hook 3 is tapered. The inward taper 10 (seen in FIG. 4) provides a low profile at the hook end of the tool. This taper provides better visibility of the hook 3 and make the hook easier to maneuver.

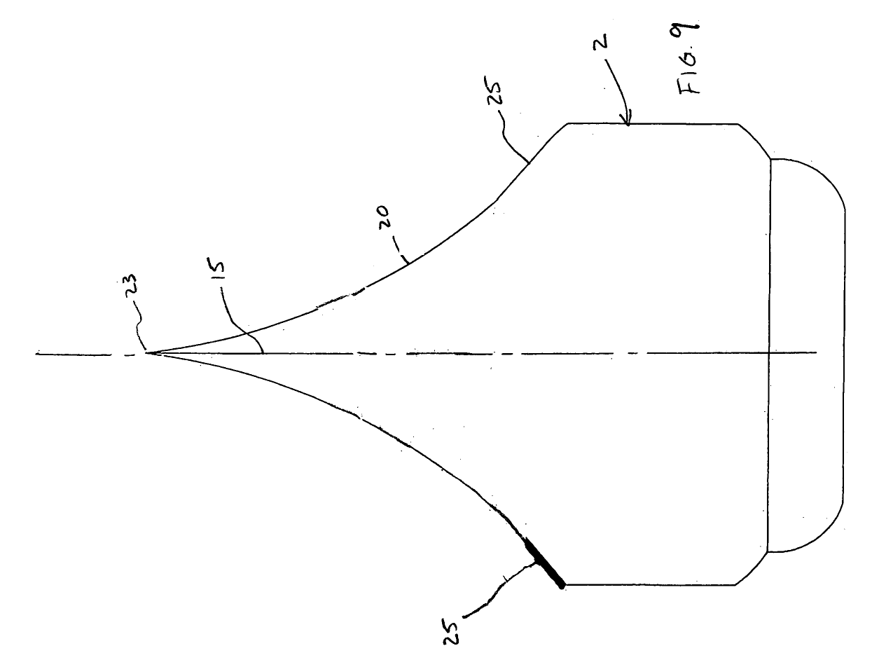

The piercing/dilating head 2 mounted to the other end of the handle can be made from a medical grade of stainless steel. As seen in FIGS. 5-7, the piercing/dilating head 2 that has been illustrated is essentially in the form of a body of revolution, but incorporates flattened, sculpted sections 11 around its circumference 12. The outer diameter of the illustrated piercing/dilating head 2 is wider than the width dimension of the handle 9 near the head, and the sculpted sections 11 help to prevent the tool from rolling when placed onto a surgical tray or other flat surface during the procedure.

The illustrated piercing/dilating head 2 has a central axis 15. In the illustrated embodiment of the invention, the axis is co-linear with an axis 17 of the handle 9 (FIG. 4). The tip 23 of the piercing/dilating head 2 that is used to make the skin opening 8 is on the axis, and is very sharp. FIG. 9 shows a dilating surface 20 on the piercing/dilating head that extends away from the tip along a curve about the axis. In the illustrated embodiment of the invention, the dilating surface 20 takes the form of a curve rotated about the axis 15 of the piercing/dilating head. In the illustrated embodiment of the invention, the curve of the dilating surface 20 bends away from the axis of the piercing/dilating head 20 as it moves away from the tip 23.

During the procedure, the tip 23 is positioned at the desired skin opening 8 location. Downward force is applied and the skin opening 8 is created. As downward force continues to be applied, the skin opening 8 begins to stretch open as the dilating surface 20 passes through the skin opening 8 and below the skin layer 6. It is preferred that the surgeon continues to advance the piercing/dilating head until a stopping surface 25 on the piercing/dilating head makes contact with the skin layer 6. In the illustrated embodiment of the invention, the stopping surface 25 begins at the lowermost edge of the dilating surface, at a distance around 0.4″ below the tip. Preferably, the stopping surface is angled at an angle of at least about 30° with respect to the axis. The stopping surface illustrated in FIG. 9 form is an angle of about 50° with respect to the central axis 15 of the piercing/dilating head. When the stopping surface comes into contact with the skin layer 6, it is preferred that the surgeon withdraw the piercing/dilating head 2 from the dilated hole.

The hook 3 illustrated in FIG. 8 is made from a medical grade of stainless steel wire. In the illustrated embodiment of the invention, the wire is round and has a diameter of about 0.039″. In use, the vein 7 is captured in a gap 27 in the hook. The width of the illustrated gap 27 is approximately 0.015″. The distance from a rounded surface 28 on the distal edge of the hook to a blunt tip 30 is approximately 0.137″. Although these dimensions are the preferred dimensions, other similar dimensions may also work.

The rounded surface 28 on the illustrated hook 3 provides a surface that can be safely passed below the skin layer 6 through the skin opening 8 without damaging underlying structures. Once the hook 3 is below the skin layer 6, the handle 9 can be slightly rotated to engage the vein, as shown in FIG. 2. The blunt tip 30 on the hook is unlikely to tear through or cut the vein 7.

The hook can then be withdrawn by pulling back on the handle 9. As the hook 3 is withdrawn, the vein 7 falls into the gap 27. The blunt end 30 of the hook 3 should not cut or tear the surrounding structures as the hook and vein are withdrawn. The hook 3 and captured vein 7 can be withdrawn through the skin opening 8 as shown in FIG. 3.

Once the vein 7 is exposed, the surgeon may remove the varicose vein segment and ligate the exposed ends of the vein following removal. Thereafter, the ligated portions of the vein 7 will return back through the incision due to the elastic nature of the vein. The dilated hole will then partially close and a tape strip can be placed over the hole. Since no linear incision was made, the hole should heal quickly and with minimal scarring.

By using a single surgical tool having both a piercing/dilating head and a hook, the surgeon can perform the steps of creating the hole, dilating the hole, engaging the vein, and withdrawing the vein all with the same tool. Further, the hole that is created should be smaller than a longitudinal incision and should heal more quickly and leave less of a scar. Accordingly, the present invention may permit the overall surgery time to be reduced, with associated benefits to the patient in terms of reduced recovery time, less scarring and medical cost savings.

Having thus described a preferred embodiment of a tool for treating varicose veins, it should be apparent to those skilled in the art that certain advantages of the above described system have been achieved. It should also be appreciated that various modifications, adaptations, and alternative embodiments thereof may be made within the scope and spirit of the present invention. The invention is defined by the following claims.

1) A surgical tool for treating varicose veins comprising:
an elongate handle with opposed ends;
a hook at one end of the handle, the hook having a gap of approximately 0.015 inches;
a piercing/dilating head at the other end of the handle, the piercing/dilating head having a bulbous shape and an axis, a sharp tip on the axis, and a dilating surface that extends along a curve about the axis.

2) A surgical tool as recited in claim 1, in which the handle tapers inwardly toward the hook.

3) A surgical tool as recited in claim 1, in which the handle comprises alternating sections of relieved surfaces and surfaces with polygonal cross-sections.

4) A surgical tool as recited in claim 1, in which the piercing/dilating head includes sculpted sections that help to prevent the tool from rolling on flat surfaces.

5) A surgical tool as recited in claim 1, in which the piercing/dilating head has an outer diameter that is wider than the width dimension of the handle near the head.

6) A surgical tool as recited in claim 1, in which the piercing/dilating head includes a stopping surface that is angled at an angle of at least about 30° with respect to the axis.

7) A surgical tool as recited in claim 1, in which the axis of the piercing/dilating head is co-linear with an axis of the handle.

8) A surgical tool as recited in claim 1, in which the portion of the piercing/dilating head is essentially in the form of a body of revolution.

9) A method for treating a varicose vein, comprising the steps of:
piercing the patient's skin and dilating the resulting opening with the piercing/dilating head of a tool as recited in claim 1; and
extracting the varicose vein with the hook of the tool.

10) A method for treating a varicose vein, comprising the steps of:
using a tool to pierce the patient's skin at a selected location;
using the same tool to dilate the resulting opening without making a longitudinal incision; and
using the same tool to extract the varicose vein from the patient.

11) A method for treating a varicose vein, comprising the steps of:
providing a tool that has an elongate handle with opposed ends, a hook at one end of the handle, and a bulbous piercing/dilating head at the other end of the handle;
using the piercing/dilating head on the tool to pierce the patient's skin at a selected location;
using the same piercing/dilating head to dilate the resulting opening without making a longitudinal incision; and
using the hook on the tool to extract the varicose vein from the patient.
